(19) United States
(12) Patent Application Publication
(10) Pub. No.: US 2015/0284668 A1
(43) Pub. Date: Oct. 8, 2015

(54) Cyclic Microfluidic Chip and Method of Using the Same

(71) Applicant: National Tsing Hua University, Hsin Chu City (TW)

(72) Inventors: Yong-Yu HSU, Keelung City (TW); Ming-Yan CHEN, Sanxing Township (TW); Kuo-Wei CHANG, Zhudong Township (TW); Tse-Shao CHEN, Puli Township (TW); Kang-Yun LEE, Songshan Dist. (TW); Han-Pin KUO, Guishan Township (TW); Yao-Fei CHAN, New Taipei City (TW); Lu-Wei KUO, Keelung City (TW); Cheng-Hsien LIU, Hsinchu City (TW)

(21) Appl. No.: 14/505,420

(22) Filed: Oct. 2, 2014

(30) Foreign Application Priority Data
Apr. 8, 2014 (TW) 103112908

(51) Int. Cl. C12M 3/06; C12M 1/00

(52) U.S. Cl. CPC C12M 23/16; C12M 23/38; C12M 45/07; C12M 45/00

(57) ABSTRACT

The present invention related to a cyclic microfluidic chip that comprises a substrate and a top cover. The substrate having a surface that provides a chamber providing location of a first cell and having a first microchannel, a second microchannel being wrapped around the outside of the chamber and comprising an ECM inlet and an ECM outlet; and a third microchannel being wrapped around the outside of the second microchannel and comprising an cell inlet and an cell outlet to provide a second cell input and output respectively. The top cover comprises a fourth microchannel to provide a medium input and a medium output.

### CROSS-REFERENCE TO RELATED APPLICATION

The application claims the benefit of TAIWAN patent application of Serial No 103112908, filed on Apr. 8, 2014, which is herein incorporated by reference.

### TECHNICAL FIELD

The present invention generally relates to a microfluidic chip, more particularly, to an especially a cyclic microfluidic chip provided for tissue culture.

### BACKGROUND OF RELATED ART

With advantage of the medical approach, the cure rates of many diseases are getting higher, however, cancer deeply affects human being. Treatment which combines traditional strategies and targeted therapy is considered as the new methodology to cure cancer. Literature indicates that when tumor grows over 2 mm in diameter, it would release the angiogenic factors, which would induce the new blood vessel grows toward the tumor, due to hypoxia. Therefore, using a cell based systems to inhibit the angiogenesis of tumor had become the new strategy of cancer treatment. In medical research, in vivo test would be with many restrictions, for instance, high cost, legal issues and ethical controversies. MEMS technology, which not only can be used to reconstruct the micro environment around tissue and reappear the function of tissue in vitro to make the research more easy and meaningful on biology, but also predict the best treatment effect for medications test to reduce the cost of clinical practice.

General cell cultures use planar substrates such as glasses or Petri dishes. However, the cost of the conventional tool is high, and it is too complicated under operation. General cell cultures are static type and it is unlikely to rebuild dynamic environments and physical stresses in vivo system. Further, general culture is not applicable for monolayer cells, and unusual features would be appeared such as drug resistance. On the contrary, cell culture using three-dimension substrates present various efficient data, phenomena and pathology. In recent research, three-dimension substrates not only create dimension gradient, but also create micro environments that is similar with the real cell environments.

Microfluidic system has many advantages, such as low consumption, low cost, less sample and reagent, and so on. Besides, the microfluidic system has the property of stable laminar flow that allows the user to control fluids factors precisely by designing microfluidic system.

In order to solve the problem of prior art, the present invention provides a novel microfluidic chip to rebuild environments of tumor cells that induce blood vessels grow towards the tumor cell. Further, it would provide more information for tumor research and therapy.

### SUMMARY

An object of the present invention is to provide a cyclic microfluidic chip that would be applicable to all tumor cell culture widely.

Another object of the present invention is to provide an operation for chip to define a cell pattern and provide precise location in order to rebuild micro environments in vitro.

Another object of the present invention is to provide a dynamic fluid with a medium with predetermined flow rate for inputting and outputting so as to raise survival rate of cells and provide more nutrition to cells sufficiently.

Yet another the present invention provides a novel micro co-culture system which is efficient and adjustable biologic apparatus to discuss the reaction of cells each other or between cells and ECM for neovascularization, so as to provide more information for tumor research and therapy

According to an aspect of the invention, it proposes a cyclic microfluidic chip provided for tissue culture. The cyclic microfluidic chip comprises a substrate and a top cover. The substrate comprises a surface which comprising a chamber, a first microchannel, a second microchannel and a third microchannel. The chamber, formed on the surface of the substrate, which is used to place a first cell, and the first microchannel, formed on a side of the chamber, which is used to provide the first cell to flow into the chamber. The second microchannel, wrapped around the outside of the chamber, which comprises an ECM (extracellular matrix) inlet for inputting ECM, and an ECM outlet that for outputting the ECM. The third microchannel, wrapped around the outside of the second microchannel, which comprises an inlet for inputting a second cell, and an outlet for outputting the second cell. The top cover, covered on the substrate, which comprise a fourth microchannel for inputting or outputting medium with a predetermined rate.

According to another aspect of the invention, the first microchannel and the fourth microchannel include a medium with predetermined flow rate for inputting and outputting so that the apparatus has dynamic fluid.

### BRIEF DESCRIPTION OF THE DRAWINGS

The components, characteristics and advantages of the present invention may be understood by the detailed description of the preferred embodiments outlined in the specification and the drawings attached.

FIG. 1 illustrates an exploded view of a cyclic microfluidic chip according the embodiment of the present invention.

FIG. 2 illustrates a perspective view of a cyclic microfluidic chip according the embodiment of the present invention.

FIG. 3 illustrates a top view and a sectional view of a cyclic microfluidic chip according the embodiment of the present invention.

FIG. 4 illustrates a pattern of a tumor cell A546 and fibroblast 3T3 via dielectrophoresis force according the embodiment of the present invention.

FIG. 5 illustrates a top view of dynamic fluid of a cyclic microfluidic chip according the embodiment of the present invention.

FIG. 6 illustrates a top view of pores between microchannels of a cyclic microfluidic chip according the embodiment of the present invention.

FIG. 7 illustrates a flow chart of the operation of a cyclic microfluidic chip according the embodiment of the present invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

### First Embodiment

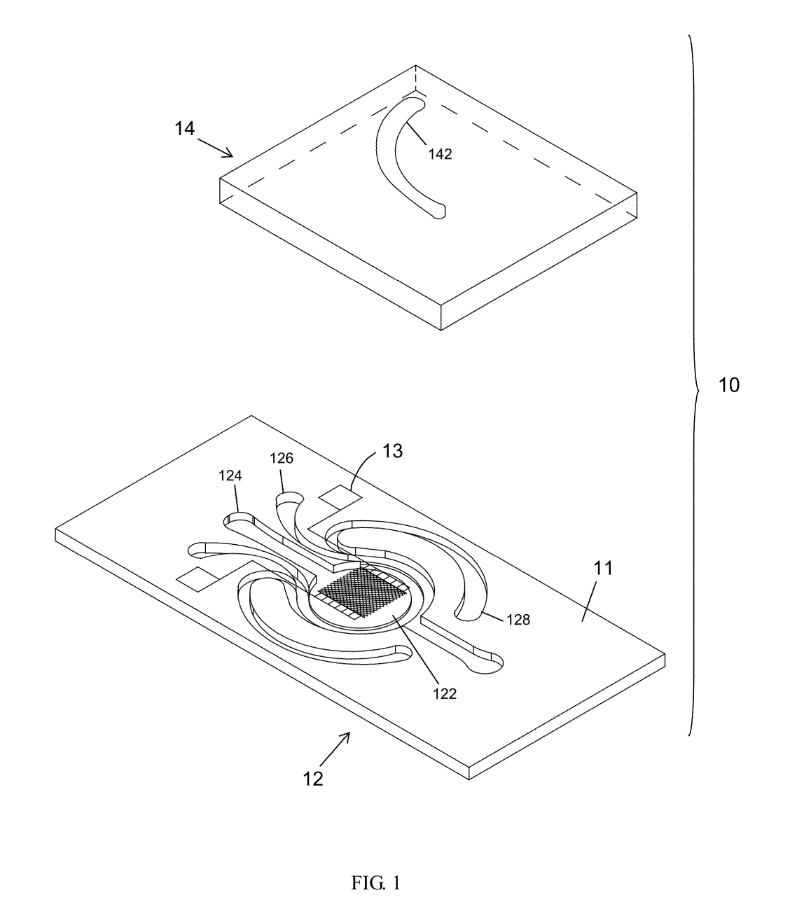

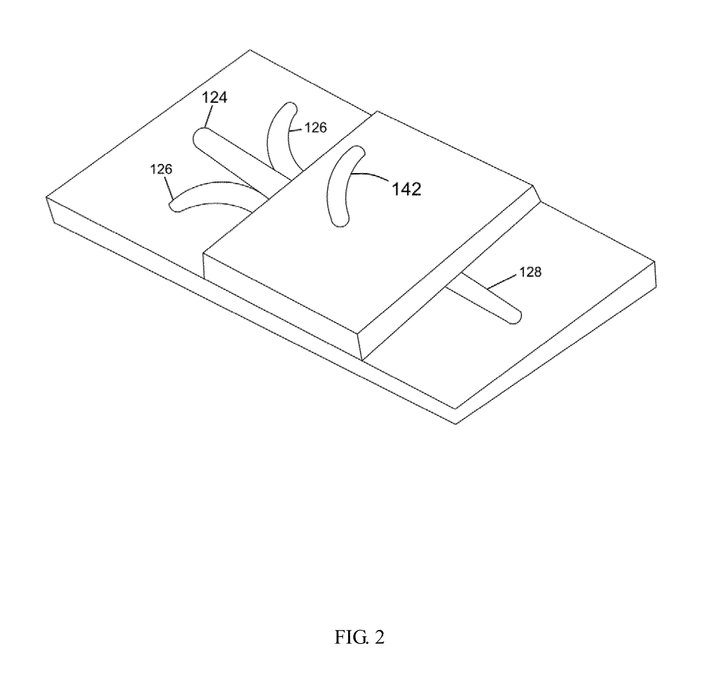

FIG. 1 and FIG. 2 show a perspective view of a cyclic microchannel chip according to one embodiment of the present invention. A cyclic microchannel chip (hereinafter referred to as the apparatus 10) includes a substrate 12 comprising a surface 11. The surface 11 has a chamber 122, a first microchannel 124, a second microchannel and a third microchannel 128. The chamber 122, formed on the surface 11 of the substrate 12, is used to place a first cell 30. The chamber 122 further includes the first microchannel 124, which is formed at a side of the chamber 122, is used to provide the first cell 30 to flow into the chamber 122. The second microchannel 126, wrapped around the outside of the chamber 122, has an ECM (extracellular matrix) inlet and an ECM outlet for inputting the ECM and outputting the same respectively. The third microchannel 128 is wrapped around the outside of the second microchannel 126. The third microchannel 128 includes an inlet for a second cell 40 flowing into the third microchannel 128, and two outlets for the second cell 40 flowing out through the third microchannel 128. The top cover 14, covered on the substrate 12, which includes a fourth microchannel 142 for a medium flowing into the chamber 122 or flowing out from the chamber 122. In another embodiment, the fourth microchannel 142 is provided to the first cell 30 to flow into the chamber 122.

In one embodiment, the length of the top cover 14 is smaller than the substrate 12. The chamber 122 has electrodes 13 in series connection. Based on the fact that any material has dielectric constant, respectively, it could be arranged with particular patterns by introducing an external electric field. In a preferred embodiment, cells are formed with particular patterns on a glass by using dielectrophoresis force (DEP).

In another embodiment, the chamber 122 further has an optical tweezer (not shown) that could control microparticles, such as cells or microorganisms, by reaction generated from single laser beam (or other light source) and momentum transfer of photon. On nondestructive conditions, optical tweezers could control cells randomly, further, arrange the cell patterns. In another embodiment, optoelectronic tweezers, that is combined with optical tweezer and dielectrophoresis force, control the distribution of electric field by operable light pattern and control the microparticles by dielectrophoresis force on an amorphous silicon material. In another embodiment, the chamber 122 further includes a microfluidic device (not shown) for cell culture by modulating flow rate. However, the above example of the chamber 122 is not limited to the configuration of the inner scope, classification or type, and other configuration, or any combination is still within the scope of the present invention.

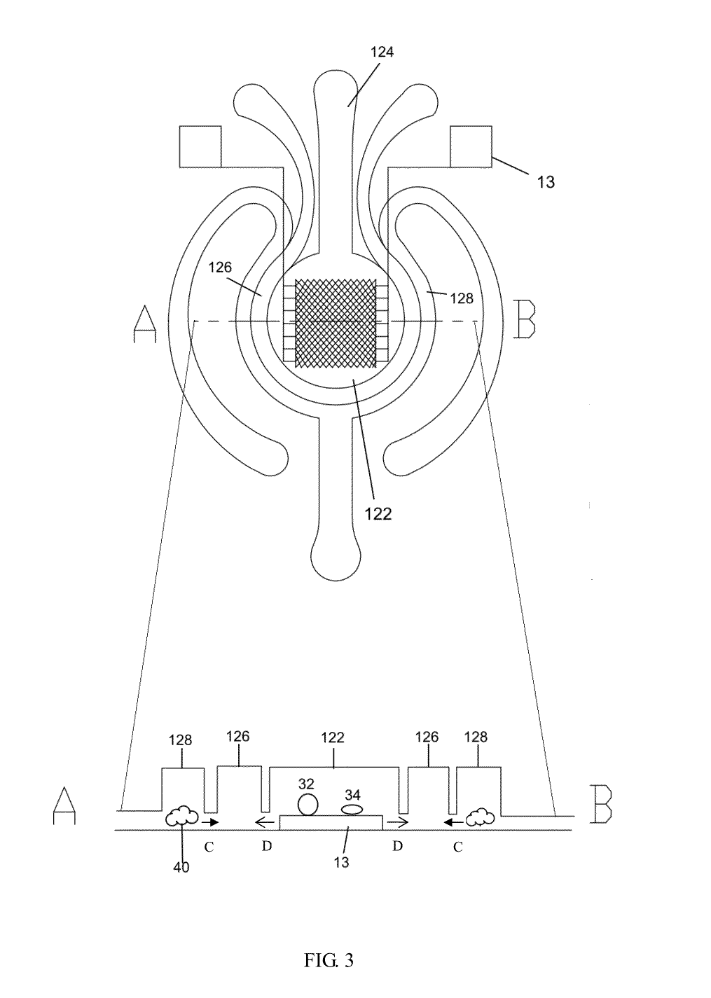

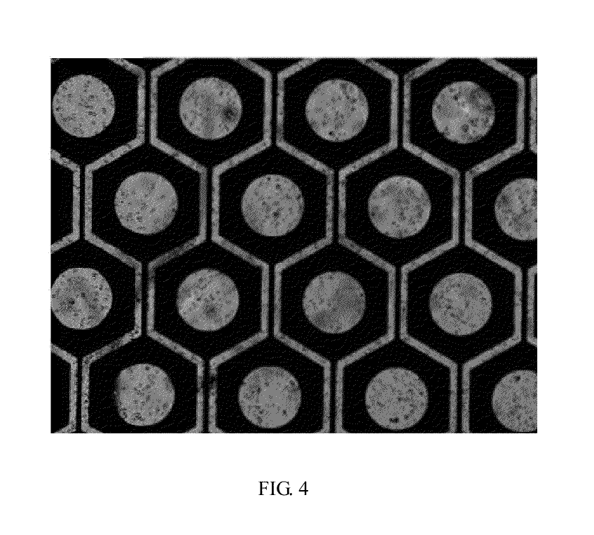

FIG. 3 shows a front view and cross-sectional view of a cyclic microfluidic chip of one embodiment. In an embodiment, the first cells 30 includes lung tumor cell (e.g. A546) 32 and fibroblast (e.g. 3T3) 34, the second cells 40 may include but be not limited to human umbilical vein endothelial cell (HUVECs). After culturing the above mentioned cells to sufficiency respectively, lung tumor cell 32 and fibroblast 34 are forced to flow into the chamber 122 via the first microchannel 124 by spring pump and attach on the top of the substrate 12. The medium flows into the chamber 122 through the fourth microchannel 142. Subsequently, parameter of positive DEP and negative DEP are set, for instance, the frequency value is set around 1 MHz and 1 kHz respectively corresponding to Vpk−pk=5V, so as to arrange cell patterns. As shown in FIG. 4, the island pattern is generated from lung tumor cell 32 and fibroblast 34 by using dielectrophoresis force. The type (or shape) of cell patterns depend on not only the arrangement of the electrodes 13 on the substrate 12 but also physics parameters of the electrodes. After the lung tumor cell patterns arrangement is completed, the lung tumor cells begin to secrete angiogenic factor which will move towards the second microchannel 126 (shown as arrow D). After that, neovascularization would be generated from the angiogenic factor and HUVECs 40 in the second microchannel 126 when HUVECs 40 are injected into the third microchannel.

In one embodiment, the chamber 122 may include but be not limited to a cyclic shape which could enhance the contact area with the third microchannel 126. By using the apparatus 10, we could discuss the directivity of chemical factor released from tumor cells whether or not. In addition, the tumor cells positioned in the chamber 122 and drugs injected into the second microchannel 126 could apply to multidirectional test.

In one embodiment, the material between microchannels is polymer membrane which is soft and flexible material, preferably, the polymer membrane is composed of but not limited to polydimethylsiloxane (PDMS). According to the experiments and simulating data, they indicate that the control of the flow rate of the microchannels and pores between the microchannels would favor the growth for neovascularization. Collagen or other biocompatibility materials could enhance the attachment between the cells and the microchannels. In one embodiment, collagen not only enhances the attachment of the cells but also acts as a holder for cells growing.

### Second Embodiment

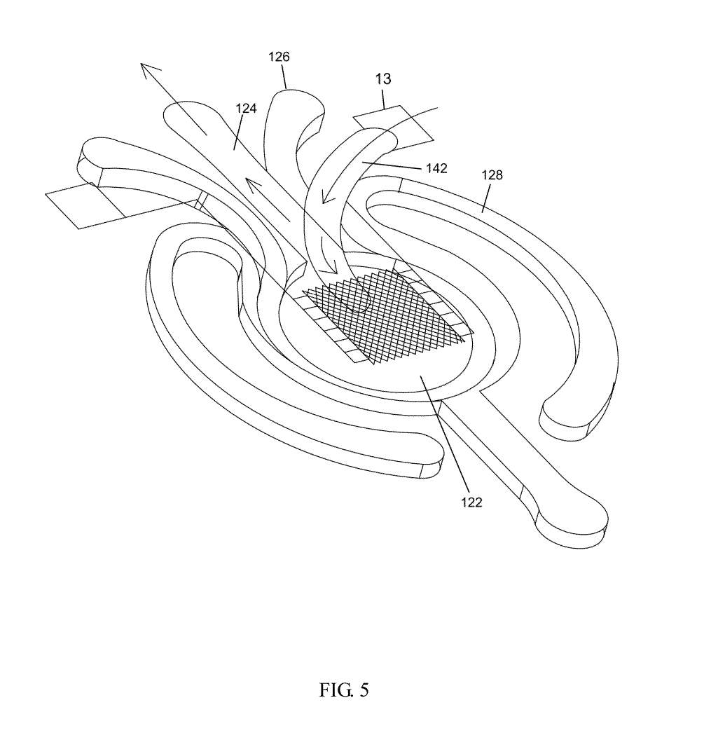

FIG. 5 shows dynamic fluid according to one embodiment of the present invention. In one embodiment, the first microchannel 124 and the fourth microchannel 142 respectively acts as medium inlet and outlet with predetermined flow rate. In other hand, the fourth microchannel 142 could act as medium inlet and outlet with predetermined flow rate simultaneously. After cells flow into the chamber 122 via the fourth microchannel 142 and attach on the surface of the substrate 12, the medium flow into the chamber 122 via the first microchannel 124 for providing nutrition to the cells. In the embodiment, the cells include any culturable cell. In culturing process, metabolites, poison or non-fresh medium could flow out from the first microchannel 124 or the fourth microchannel 142, meanwhile, fresh medium could flow into the chamber 122 via the fourth microchannel 142. In aforementioned culturing process, the apparatus with predetermined flow rate may make sure that the cells obtain fresh nutrition so as to raise the survive of the cells. The flow rate would be modulated based on the require conditions.

In one embodiment, the material between microchannels is polymer membrane which is soft and flexible material, preferably, the polymer membrane is composed of but be not limited to polydimethylsiloxane (PDMS). According to the experiments and simulating data, they confirm that the control of the flow rate of the microchannels and pores between the microchannels would favor the growth for neovascularization. Collagen or other biocompatibility materials could enhance the attachment between cells and microchannels. In one embodiment, collagen not only enhances the attachment of cells but also acts as a holder for cells growing.

### Third Embodiment

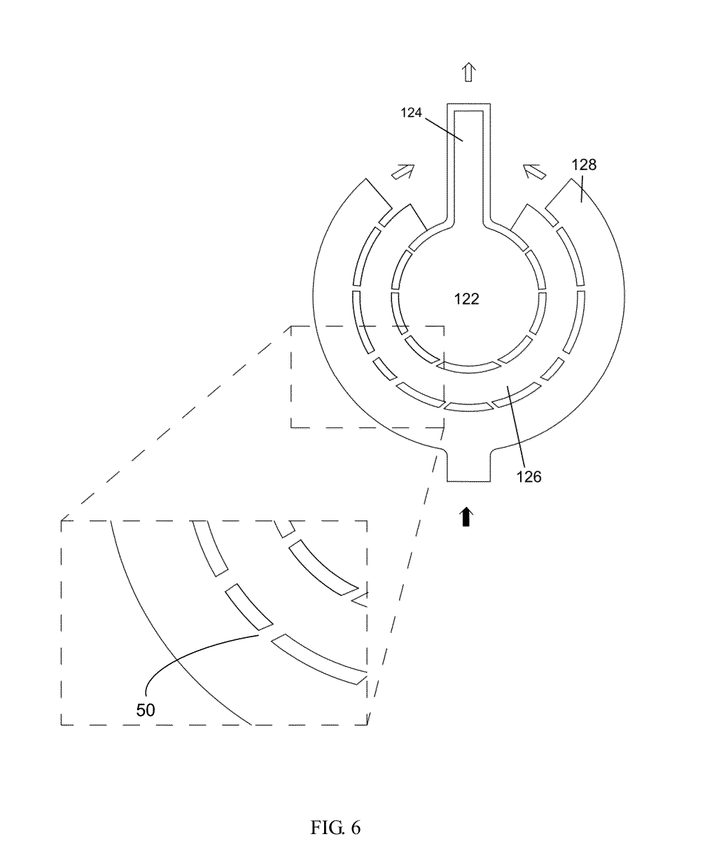

FIG. 6 shows a top view of the pores between microchannels of a cyclic microfluidic chip according the embodiment of the present invention. In the embodiment, fibroblast and the first cell culture medium (not shown) are injected into the third microchannel 128, the first cell culture medium is EBM-2 Basal Medium. Then, second cell culture medium with fetal bovine serum (FBS) (not shown) are injected into the chamber 122. After 16 hours, fibroblast moved towards the second microchannel 126 and attached on the wall between the second microchannel 126 and the third microchannel 128. According to the embodiment, it clearly figures out to realize that the movement of cells towards the pores 50 between the microchannels, results from influence of concentration gradient, would improve the growth for neovascularization.

In one embodiment, the material between microchannels is polymer membrane which is soft and flexible material, preferably, the polymer membrane is composed of but be not limited to polydimethylsiloxane (PDMS). According to the experiments and simulating data, they indicate that the control of the flow rate of the microchannels and pores between the microchannels would improve the growth for neovascularization. Collagen or other biocompatibility materials could enhance the attachment between cells and microchannels. In one embodiment, collagen not only enhances the attachment of cells but also acts as a holder for cells growing.

### Fourth Embodiment

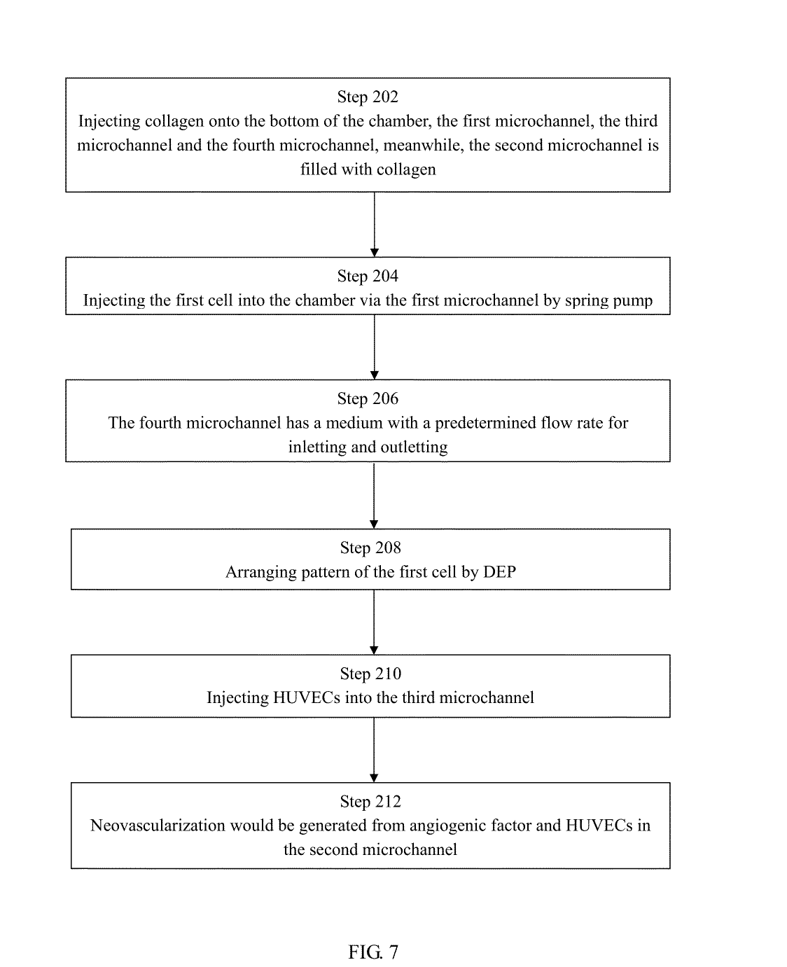

FIG. 7 shows a flow chart of the operation of a cyclic microfluidic chip according the embodiment of the present invention.

Step 202: Injecting collagen onto the bottom of the chamber 122, the first microchannel 124, third microchannel 128 and fourth microchannel 142, meanwhile, the second microchannel 126 is filled with collagen that used to hold and support the cells.

Step 204: Injecting the first cell 30 into the chamber 122 via the first microchannel 124 by spring pump. In the embodiment, the first cell 30 includes lung tumor cell 32 and fibroblast 34.

Step 206: The fourth microchannel 142 is provided medium to inlet or outlet with predetermined flow rate, that would make sure the first cell 30 to obtain fresh nutrition, as shown in FIG. 5. The flow rate could be set based on the required condition.

Step 208: Arranging the cell patterns by DEP, as shown in FIG. 4. In the embodiment, Subsequently, parameter of positive DEP and negative DEP are set, for instance, the frequency value is set around 1 MHz and 1 kHz respectively corresponding to Vpk−pk=5V.

In another embodiment, the chamber 122 further includes an optical tweezer (not shown) that could control microparticles, such as cells or microorganisms, by reaction generated from single laser beam (or other light source) and momentum transfer of photon. On nondestructive conditions, optical tweezers could control cells randomly, further, arrange cell patterns. In another embodiment, the combination of the optical tweezer and dielectrophoresis force are introduced to control the distribution of electric field by operable light pattern and control the microparticles by dielectrophoresis force on the amorphous silicon material. In another embodiment, the chamber 122 further includes a microfluidic device (not shown) for cell culture by modulating flow rate. However, the above example of the chamber 122 is not limited to the configuration of the inner scope, classification or type, and other configuration, or any combination is still within the scope of the present invention.

Step 210: Injecting the second cell 40 into the third microchannel 128. In the embodiment, the second cell 40 may comprise but be not limited to human umbilical vein endothelial cell (HUVECs).

Step 212: Neovascularization would be generated from angiogenic factor and HUVECs 40 in the second microchannel 126 when HUVECs 40 injected into the third microchannel 128.

In one embodiment, the material between microchannels is polymer membrane which is soft and flexible material, preferably, the polymer membrane is composed of but not limited to polydimethylsiloxane (PDMS). According to the experiments and simulation data, they indicate controlling the flow rate of the microchannels and pores between the microchannels would flavor the growth for neovascularization. Collagen or other biocompatibility materials could enhance the attachment between cells and microchannels. In one embodiment, collagen not only enhances the attachment of cells but also act as a holder for cells growing.

In the specification, the substrate includes silicon photoconductivity material, amorphous silicon conductivity material or other polymer membrane, and so on. The width of different microchannels would be the same or not based on the required conditions. The size of the chamber would be modulated based on the required conditions, such as small or bigger than the microchannel, even equal to it.

In the specification, the apparatus may act as a platform for not only multi-cell but also single-cell. “Animal tissue”, “tumor cell” or “cell” may include but be not limited to cell which could release chemical factor, and from human or ones organs. The ECM may include but be not limited to collagen and fibronectin that be used to support and holder cells. The aforementioned apparatus may be applied to any cell culture widely, not limited to tumor cell culture. Furthermore, the apparatus may apply to detection of cell or other organism.

If it is said that an element “A” is coupled to or with element “B,” element A may be directly coupled to element B or be indirectly coupled through, for example, element C. When the specification states that a component, feature, structure, process, or characteristic A “causes” a component, feature, structure, process, or characteristic B, it means that “A” is at least a partial cause of “B” but that there may also be at least one other component, feature, structure, process, or characteristic that assists in causing “B.” If the specification indicates that a component, feature, structure, process, or characteristic “may”, “might”, or “could” be included, that particular component, feature, structure, process, or characteristic is not required to be included. If the specification refers to “a” or “an” element, this does not mean there is only one of the described elements.

The foregoing descriptions are preferred embodiments of the present invention. As is understood by a person skilled in the art, the aforementioned preferred embodiments of the present invention are illustrative of the present invention rather than limiting the present invention. The present invention is intended to cover various modifications and similar arrangements included within the spirit and scope of the appended claims, the scope of which should be accorded the broadest interpretation so as to encompass all such modifications and similar structures.

What is claimed is:

1. A cyclic microfluidic chip for tissue culture, comprising:
a substrate having a surface comprising a chamber, a first microchannel, a second microchannel and a third microchannel formed thereon, wherein
said chamber formed on said surface of said substrate to place a first cell, and said first microchannel being formed on a side of said chamber to allow said first cell to flow into said chamber,
said second microchannel being wrapped around outside of said chamber and comprising an ECM (extracellular matrix) inlet for inputting ECM, and an ECM outlet for outputting the ECM,
said third microchannel being wrapped around outside of said second microchannel comprising an cell inlet for inputting a second cell, and an cell outlet for outputting said second cell; and
a top cover, covered on said substrate, comprises a fourth microchannel for inputting or outputting medium with a predetermined rate.

2. The cyclic microfluidic chip of claim 1, wherein said chamber comprises two electrodes for cell patterning by biasing.

3. The cyclic microfluidic chip of claim 1, wherein said chamber further comprises an optical tweezers device for cell patterning.

4. The cyclic microfluidic chip of claim 1, wherein said chamber further comprises a microfluidic device for cell patterning.

5. The cyclic microfluidic chip of claim 1, wherein said shape of the chamber comprises cyclic and spheroid.

6. The cyclic microfluidic chip of claim 1, wherein the ECM in said second microchannel comprises a collagen for holding said second cell.

7. The cyclic microfluidic chip of claim 1, wherein said collagen coated on said substrate is provided to enhance an adhesion between said first cell and said substrate.

8. The cyclic microfluidic chip of claim 1, wherein said first cell comprises a tumor cell and a fibroblast.

9. The cyclic microfluidic chip of claim 8, wherein said tumor cell comprises an organelle from animals.

10. The cyclic microfluidic chip of claim 1, wherein said second cell comprises an endothelium.

11. A cyclic microfluidic chip for tissue culture, comprising:
a substrate having a surface comprising a chamber, a first microchannel, a second microchannel and a third microchannel formed thereon, wherein
said chamber formed on said surface of said substrate to place a first cell, and said first microchannel being formed on a side of said chamber for inputting or outputting medium with a predetermined rate,
said second microchannel being wrapped around outside of said chamber and comprising an ECM (extracellular matrix) inlet for inputting ECM, and an ECM outlet for outputting the ECM,
said third microchannel being wrapped around outside of said second microchannel comprising an cell inlet for inputting a second cell, and an cell outlet for outputting said second cell; and
a top cover, covered on said substrate, comprises a fourth microchannel to allow said first cell to flow into said chamber.

12. The cyclic microfluidic chip of claim 11, wherein said chamber comprises two electrodes for cell patterning by biasing.

13. The cyclic microfluidic chip of claim 11, wherein said chamber further comprises an optical tweezers device for cell patterning.

14. The cyclic microfluidic chip of claim 11, wherein said chamber further comprises a microfluidic device for cell patterning.

15. The cyclic microfluidic chip of claim 11, wherein said shape of the chamber comprises cyclic and spheroid.

16. The cyclic microfluidic chip of claim 11, wherein the ECM in said second microchannel comprises a collagen for holding said second cell.

17. The cyclic microfluidic chip of claim 11, wherein a collagen coated on said substrate is provided to enhance an adhesion between said first cell and said substrate.

18. The cyclic microfluidic chip of claim 11, wherein said first cell includes a tumor cell and a fibroblast.

19. The cyclic microfluidic chip of claim 11, wherein said second cell comprises an endothelium.

20. A cyclic microfluidic chip for tissue culture, comprising:
a substrate having a surface comprising a chamber, a first microchannel, a second microchannel and a third microchannel formed thereon, wherein
said chamber formed on said surface of said substrate to place a first cell, and said first microchannel being formed on a side of said chamber for inputting or outputting medium with a predetermined rate,
said second microchannel being wrapped around outside of said chamber and comprising an ECM (extracellular matrix) inlet for inputting ECM, and an ECM outlet for outputting the ECM, and
said third microchannel being wrapped around outside of said second microchannel comprising an cell inlet for inputting a second cell, and an cell outlet for outputting said second cell.
